(19) United States
(12) Patent Application Publication
(10) Pub. No.: US 2016/0362402 A1
(43) Pub. Date: Dec. 15, 2016

(54) NOVEL COMPOUNDS HAVING ANTI-ALLODYNIC AND ANTIHYPERALGESIC ACTIVITY

(71) Applicants: CONSORZIO INTERUNIVERSITARIO NAZIONALE PER LA SCIENZA E TECNOLOGIA DEI MATERIALI, Firenze (IT); UNIVERSITA DEGLI STUDI DI FIRENZE, Firenze (IT)

(72) Inventors: Cristina NATIVI, Firenze (IT); Carla GHELARDINI, Pistoia (IT); Giancarlo LA MARCA, Firenze (IT)

(21) Appl. No.: 15/121,649

(22) PCT Filed: Feb. 27, 2014
(86) PCT No.: PCT/IB2014/059289

§ 371 Date: Aug. 25, 2016

(51) Int. Cl. C07D 409/12; A61K 31/282; A61K 31/4025; C07D 339/04; A61K 31/381

(52) U.S. Cl. CPC C07D 409/12; C07D 339/04; A61K 31/381; A61K 31/4025; A61K 31/282

(57) ABSTRACT

The present invention relates to molecules of formula (I) where R1=—SO₃H, —PO₃H, —PO₂(OH)₂, —OPO₂H₂, —NHSO₃H, —S(N═H)Me, SH, SR or guanidyl; R═C₁₋₄ alkyl, phenyl or 5 or 6 membered aromatic nitrogen heterocycles; n=1, 2, 3, 4 or 5; X═C=0, C(OH)H, C(OAlk)H, C═S, CH₂; Alk=C₁₋₆ alkyl linear, branched or cyclic, optionally hydroxylated or polyhydroxylated; their preparation and use as analgesics and in the treatment of pain induced by chemotherapies.

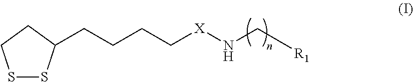

### FIELD OF THE INVENTION

The present invention relates to the field of organic compounds containing heterocycles having pharmacological efficacy as anti-hyperalgesics and anti-allodynics.

### STATE OF THE ART

The World Health Organisation (W.H.O.) defines neuropathic pain as: “An unpleasant sensation and a negative-affective emotional experience, associated with actual or potential tissue damage, or described in terms of such damage”. The International Association for the Study of Pain (IASP) defines it as: “An unpleasant sensory and emotional experience associated with actual or potential tissue damage, or described as such”.

Neuropathic pain is a significant problem in neurology in that it occurs frequently and is often disabling due to its irritating and chronic character.

Examples thereof are: post-herpetic pain, phantom limb pain which can arise after an amputation, pain present in peripheral neuropathy such as with diabetes or AIDS, so-called complex regional pain syndrome or reflex sympathetic dystrophy pain, and pain from lesions of the central nervous system. These latter can be sequelae of stroke, trauma, tumours or due to systemic diseases. In most cases, the pain often present in multiple sclerosis is of such origin. In recent years, interest has focussed on neuropathic pain induced by chemotherapy drugs (vincristine, paclitaxel, oxaliplatin, bortezomib, etc.)

The characteristics of this pain vary from patient to patient, but usually have ongoing burning or electric shock sensations; paresthesia is often present, i.e. abnormal sensations even in the areas surrounding the primary site of pain. These sensations are known as hyperalgesia, when a slightly painful stimulation in fact creates a very strong pain, and allodynia, when a non-painful stimulation, which can be simply stroking the skin or the weight of a sheet, is perceived as pain.

This type of pain does not respond well to the most common analgesics such as acetyl salicylic acid, paracetamol or the much-used non-steroidal anti-inflammatory drugs, and even morphine is only partially effective. This type of pain is difficult to cure and yet no specific treatments exist; it is one of the most frustrating problems in analgesic therapy.

The most commonly used drugs to treat this type of pain are the anticonvulsants such as gabapentin, carbamazepine and lamotrigine, lidocaine in patch form (not yet available in Italy), tramadol, tricyclic antidepressants such as amitriptyline or the better tolerated nortriptyline. Tramadol and opioid drugs are to be used with particular care because of their dependence potential, and tricyclic antidepressants can have serious side effects particularly in the elderly. If good pain control is not achieved with a single first choice drug, a combination of several drugs is justified since the molecular mechanisms acted on by the various drug categories are different.

Neuropathic pain induced by chemotherapy drugs deserves attention on its own. oxaliplatin, a platinum-based chemotherapy drug, is nowadays become a standard treatment for advanced cancer of the colon rectum. In contrast to other platinum derivatives (e.g., cisplatin), oxaliplatin causes reduced damages to kidneys, has reduced ototoxicity, and presents a mild hematic and gastrointestinal toxicity. The actual treatment-limiting issue in the usage of oxaliplatin is the development of neuropatic pain, consisting in foot/leg, hand/arm numbness combined with paresthesia, dysesthesia and pain. All these symptoms may become highly invalidating for patients, severely affecting their quality of life.

Unfortunately, repeated treatments with oxaliplatin may cause chronic neuropathic pain that is very often responsible for therapy interruption. A truly effective pharmacological treatment for this kind of neuropathic pain is presently lacking and clinical trials have shown that the prophylactic or therapeutic effects of anti-hyperalgesic drugs for repeated oxaliplatin treatments are completely inconclusive. In addition, the fundamental requirement of a drug to be employed for this kind of neuropathic pain, that is, to not contrast the anti-tumor activity of the chemotherapic drug, should not be underestimated.

Infusion of calcium and magnesium afforded good results against this kind of pain and patients treated this way did not develop acute neuropathic pain symptoms. Unfortunately, however, trials of this treatment have been interrupted, because patients receiving calcium and magnesium showed stronger tumor-related side effects.

“Radical scavengers” have been employed against neuropathic pain from chemotherapic drugs. Recent studies, however, reported that patients receiving Amifostine had to interrupt treatment because of hypotension issues.

Glutathione gave good results as a neuroprotecting drug when used for reducing cisplatin accumulating in the “root dorsal ganglia” of treated patients, but a decrease of the antitumor activity has also been observed.

Anticonvulsant drugs, such as Carbamazepine, have also been employed in patients treated with oxaliplatin, but without any beneficial effects in terms of pain relief. Other drugs are currently under investigation, but trials are not statistically significant at present.

WO2012/067947 describes lipoic acid derivatives for usage in the treatment of ischemic damage; among the examples, the compounds N—(R)-lipoyl-β-alanine, N—(R)-lipoyl-β-taurine, N—(R)-lipoyl-aminoethylphosphonic acid and N—(R)-lipoyl-aminoethyl-dihydrogenphosphate are described.

EP1371640, EP1547590 JP2011-195516 describe, as precursors of metal complexes, the sodium, potassium, calcium and magnesium salts of N-(α)-lipoyl-aminoethanesulfonic acid, the sodium salt of N-(α)-lipoyl-6-aminohexanoic acid, the sodium salt of N-(α)-lipoyl-γ-amino-n-butanoic acid and the sodium salt of N-(α)-lipoylglycine. These metal complexes are described as possessing inhibition activity toward tyrosinase. WO2011/080725 describes compounds possessing analgesic or anti-hyperalgesic activity.

There is hence an evident need to provide molecules which are at least alternatives to those currently available, which are effective in controlling neuropathic pain and possibly present fewer side effects such as dependency or behavioral changes.

### SUMMARY OF THE INVENTION

The object of the present invention are compounds of formula (I)

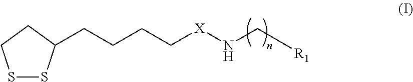

wherein

R1=—SO₃H, —PO₃H, —PO₂(OH)₂, —OPO₂H₂, —NHSO₃H, —S(N═H)Me, —COOH, —SH, —SR or guanidyl;

R═C₁₋₄ alkyl, phenyl or 5 or 6 membered aromatic nitrogen heterocycles;

n=1, 2, 3, 4 or 5;

X═C═O, C(OH)H, C(OAlk)H, C═S, CH₂

Alk=C₁₋₆ alkyl linear, branched or cyclic, optionally hydroxylated or polyhydroxylated, which are meant to include all possible optical isomers such as enantiomers and/or diastereoisomers, mixtures thereof, either as racemes or in various ratios, and inorganic or organic salts (pharmaceutically acceptable);

excluding compounds N—(R)-lipoyl-β-alanine, N—(R)-lipoyl-β-taurina, N—(R)-lipoyl-aminoethylphosphonic acid, N—(R)-lipoyl-aminoethyldihydrogenphosphate, the sodium, potassium, calcium and magnesium salts of N-(α)-lipoyl-aminoethanesulfonic acid, the sodium salt of N-(α)-lipoyl-6-aminohexanoic acid, the sodium salt of N-(α)-lipoyl-γ-amino-n-butanoic acid and the sodium salt of N-(α)-lipoylglycine.

Subject-matter of the present invention are also compounds for use as medicaments, said compounds of formula (I)

wherein

R1=—SO₃H, —PO₃H, —PO₂(OH)₂, —OPO₂H₂, —NHSO₃H, —S(N═H)Me, —COOH, —SH, —SR or guanidyl;

R═C₁₋₄ alkyl, phenyl or 5 or 6 membered aromatic nitrogen heterocycles;

n=1, 2, 3, 4 or 5;

X═C═O, C(OH)H, C(OAlk)H, C═S, CH₂

Alk=C₁₋₆ alkyl linear, branched or cyclic, optionally hydroxylated or polyhydroxylated, which are meant to include all possible optical isomers such as enantiomers and/or diastereoisomers, mixtures thereof, either as racemes or in various ratios, and inorganic or organic salts (pharmaceutically acceptable);

excluding of compounds N—(R)-lipoyl-β-alanine, N—(R)-lipoyl-β-taurine, N—(R)-lipoyl-aminoethylphosphonic acid and N—(R)-lipoyl-aminoethyldihydrogenphosphate.

In particular, subject-matter of the invention are compounds for use as anti-hyperalgesic and anti-allodynic; said compounds of formula (I) wherein

R1=—SO₃H, —PO₃H, —PO₂(OH)₂, —OPO₂H₂, —NHSO₃H, —S(N═H)Me, —COOH, —SH, —SR or guanidyl,

R═C₁₋₄ alkyl, phenyl or 5 or 6 membered aromatic nitrogen heterocycles;

n=1, 2, 3, 4 or 5;

X═C═O, C(OH)H, C(OAlk)H, C═S, CH₂

Alk=C₁₋₆ alkyl linear, branched or cyclic, optionally hydroxylated or polyhydroxylated, which are meant to include all possible optical isomers such as enantiomers and/or diastereoisomers, mixtures thereof, either as racemes or in various ratios, and inorganic or organic salts (pharmaceutically acceptable).

Pharmacological tests performed on a compound of formula (I) as above described have demonstrated that these molecules possess surprising antioxidant properties and that the antioxidant profile also emerged in a rat primary astrocytes cell culture (FIG. 1) in the presence of oxalilplatin. In vivo experiments have evidenced the ability of the above described compounds of formula (I) of reverting the hyperalgesia induced by the neurotoxic substance oxaliplatin.

It is therefore further subject-matter of the present invention a pharmaceutical composition comprising a compound of formula (I) and at least another ingredient pharmaceutically acceptable; preferably, the aforesaid ingredient is selected among chemotherapies (such as, for example, oxaliplatin or cisplatin).

The object of the present invention is also a process for the preparation of compounds of formula (I) from lipoic acid, the carboxylic group of which has been preferably and appropriately activated for the formation of an amidic linkage, and a compound of formula (II)

R₁—(CH₂)n-NH₂ (II)

in which

R1=—SO₃H, —PO₃H, —PO₂(OH)₂, —OPO₂H₂, —NHSO₃H, —S(N═H)Me, —COON, —SH, —SR or guanidyl;

R═C₁₋₄ alkyl, phenyl or 5 or 6 membered aromatic nitrogen heterocycles;

n=1, 2, 3, 4 or 5;

and in which, for synthetic convenience, R1 may be appropriately masked or protected, as known to a skilled practitioner.

The object of the present invention are also the synthetic intermediates for the preparation of compounds of formula (I).

### BRIEF DESCRIPTION OF THE FIGURES

FIG. 1. Evaluation of the antioxidant profile of ADM-12 in a rat primary astrocytes cell culture *P<0.01 with respect to the untreated control and ̂P<0.01 with respect to astrocytes treated with oxaliplatin.

### DETAILED DESCRIPTION OF THE INVENTION

Compounds of formula (I) in which the stereogenic centre deriving from lipoic acid is in the (R) or (R,S) configuration are preferred.

Compounds of formula (I) in which —R1=—SO₃H are preferred; are also preferred those compounds in which n=2, 3 or 4, and those compounds in which X═C═O.

In particular, a compound of formula (I), in which R1=—SO₃H, X═C═O and n=3 (hereafter named ADM-12), preferably as racemic mixture, is preferred.

Preferably the above described compounds are useful in the treatment of neuropathic pain, in particular neuropathic pain induced by chemotherapies. Said chemotherapies are preferably selected in the group consisting of neurotoxic drugs and more preferably within the group consisting of oxaliplatin, cisplatin, paclitaxel, vincristina, vinblastina.

For elucidating their mechanism of action, compounds according to the invention, and in particular ADM-12, have been subjected to studies, in vivo and in vitro, from which it emerged that, besides showing evident antioxidant properties, said compounds are also useful in the treatment of pain induced by the inflammation of the trigeminal nerve and also in the treatment of the “restless legs” syndrome, but also for use in the treatment of rhinitis or for use in the treatment of itch.

The compounds of formula (I) according to the invention are chemically stable in saliva and in pH conditions either acidic or alkaline. Furthermore, it has been verified that compounds of formula (I) and chemotherapies (such as oxaliplatin) are compatible when in admixtures; indeed, it has been verified that ADM-12 and oxaliplatin remain structurally unaltered when mixed in physiological solution.

It is therefore the preferred subject-matter of the present invention a pharmaceutical composition comprising a compound of formula (I) and a chemotherapic agent; the aforesaid chemotherapic agent is preferably selected in the group consisting of neurotoxic drugs and more preferably within the group consisting of oxaliplatin, cisplatin, paclitaxel, vincristina, vinblastina.

The compounds of formula (I) as above described can be preferably prepared by means of two synthetic steps in which firstly the lipoic acid is reacted with a reagent capable of activating the carboxylic acid group, for the subsequent formation of an amidic bond with a compound of formula (II)

R₁—(CH₂)n-NH₂ (II)

in which

R1=—SO₃H, —PO₃H, —PO₂(OH)₂, —OPO₂H₂, —NHSO₃H, —S(N═H)Me, —COOH, —SH, —SR or guanidyl;

R=C₁₋₄ alkyl, phenyl or 5 or 6 membered aromatic nitrogen heterocycles;

n=1, 2, 3, 4 or 5.

and in which R1, for synthetic convenience, can be appropriately masked or protected, as known to a skilled practitioner.

Preferably the compound of formula (II) is 3-aminopropan-1-sulfonic acid.

Said two steps are sufficient to obtain a compound of formula (I) in cui X═C═O.

A compound of formula (I) in which X═C(OH)H, C═S, CH₂ can be prepared from a compound of formula (I) in which X═C═O by means of known and appropriate methodologies for the transformation of the C═O bond.

Given the different lipo-hydrophilic characteristics of the two molecules, the choice of reaction conditions (solvent and accompanying agent) is not obvious, and neither is the purification of the final product. Many of the conditions generally used for similar molecules and similar reactions have not in fact led to the desired product with the desirable yields and purity.

Preferably the lipoic acid is activated by treatment with N-hydroxysuccinimide to obtain the compounds of formula (III)

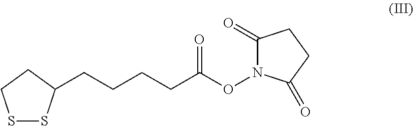 [SMILES: O=C(CCCCC1CCSS1)ON1C(=O)CCC1=O]

which include the two possible enantiomers and mixtures thereof.

The above said compounds of formula (III) are isolable and are useful intermediates for the synthesis of the compounds of formula (I) as above described.

The compounds of formula (III) have been previously described in WO2011/080725.

The compounds of formula (III) as aforedescribed can be obtained by reacting lipoic acid with N-hydroxysuccinimide in the presence of a carbodiimide (e.g. cyclohexyl carbodiimide, 4-(4,6-dimethoxy-1,3,5-triazin-2-yl)-4methylmorpholinium chloride (DMTMM), O-(7-azabenzotriazol-1-yl)-N,N,N′,N′-tetramethyluronium hexafluorophosphate (HATU), oxalyl chloride, isopropenyl chloroformate (IPCF), in a polar aprotic solvent (e.g. THF, DMF, diethyl ether, nitromethane, acetonitrile, triethylamine). Whereas adding the carbodiimide to the remaining reaction mixture is preferably carried out at a temperature of 0-5° C., the reaction mixture is then heated to ambient temperature (20-25° C.) and left to react for a time sufficient to complete the reaction (e.g. 5-6 hours).

The compounds of formula (III) as above described can then be reacted with compounds of formula (II) as above described to obtain the compounds of formula (I) as above described in which X═C═O. In particular the compounds of formula (III) can be reacted with 3-aminopropan-1-sulfonic acid to obtain a compound of formula (I) in which X═C═O, R1=—SO₃H and n=3 (ADM-12).

Preferably the reaction between the intermediates of formula (III) and compounds of formula (II) is carried out in mixtures of H2O/polar aprotic solvent (e.g. DMSO, DMF, acetonitrile, nitromethane, THF) in the presence of a base (e.g. NaHCO3, Na2CO3, triethylamine, pyridine, lutidine, DBU). Preferably the intermediate of formula (III), dissolved in a polar aprotic solvent (e.g. DMF), is slowly added to a solution of compound of formula (II) (in 0.5-1.5 hours) at a temperature between −5 and +5° C.; the reaction mixture is then left at room temperature (20-25° C.) for a time sufficient to complete the reaction (for example 12-18 ore).

The product obtained at the end of the process is extracted in an organic solvent (for example AcOEt) and, once the solvent is evaporated, is a solid and can be conveniently purified by filtration over silica gel.

The compounds of formula (I), in which X═C═O, at the end of the above described process, are obtained in the form of salts in which the cation corresponds to that of the base used in the coupling reaction with the compound of formula (II).

The present invention can be better understood in the light of the following embodiments.

Experimental Part

The following describes an example of the synthesis of a compound of the invention in a racemic mixture starting from lipoic acid and 3-aminopropan-1-sulfonic acid by means of the steps shown in the following scheme:

Synthesis of Compound 1 [Compound of Formula (III)]

To a solution of R/S lipoic acid (1 g, 4.85 mmol) and N-hydroxysuccinimide (674 mg, 5.82 mmol) in 30 mL of THF at 4° C. is slowly added a solution of cyclohexylcarbodiimide (1.2 g, 5.82 mmol in 2 mL of THF). The mixture is heated at room temperature (20-25° C.) and stirred for 5.5 hours. The solid is filtered off and the organic solvent is evaporated to give a yellow solid which is purified by crystallization (EtOAc/hexane 1:1). Compound 1 is obtained as a pure solid in 57% yield.

¹H NMR (CDCl₃): δ 1.4-2.1 (m), 2.4-2.6 (m), 2.7 (t), 2.9 (as), 3.1-3.3 (m), 3.5-3.7 (m).

Synthesis of Compound 2 (ADM-12)

To a suspension of 3-aminopropan-1-sulfonic acid (homotaurine) (1.5 g, 10.88 mmol) and NaHCO₃ in H₂O/DMF (1:1 v/v, 30 mL) is added at 0° C. a solution of compound 1 in DMF (20 mL). The mixture is heated at room temperature (20-25° C.) and stirred for 18 hours. After several washings with AcOEt, the acqueous phase is evaporated and the yellow solid is dissolved in methanol and filtered over silica gel (dichloromethane/methanol 2:1). This way, compound 2 is obtained as a pure solid with a 59% yield.

¹H NMR (D₂O): d 1.20-1.25 (m), 1.4-1.6 (m), 1.65-1.85 (m), 2.05 (t), 2.2-2.4 (m), 2.6-2.8 (m), 3.0-3.2 (m), 3.4-3.6 (m), 7.8 (bs).

Chemical Stability

ADM-12 was found to be chemically stable under the following conditions: in saliva, and at pH 1 e 10.

ADM-12 in physiological solution with oxaliplatin after 24 e 48 hours at room temperature and at 37° C. does not show signal variations at ¹H NMR and in mass spectra; likewise, oxaliplatin does not show signal variations; from these evidence is inferred that both compounds would not be affected by structural variations, when present in a single composition.

Pharmacological Tests

In Table 1 are reported the effects of the compounds in the Nitro Blue Tetrazolium (NBT) oxidation test. Experiments have been carried out according to the method described by Ciuffi et al., 1998. The text consists in the production of superoxide anion by means of the reaction between hypoxanthine (600 mM) and xanthine oxidase (10 mU/ml). The antioxidant profile of the above described compounds has been evaluated monitoring the oxidation kinetics of nitro blue tetrazolium (NBT, 10 mM) in the presence of increasing concentration of each single compound. The antioxidant activity has been measured spectrophotometrically at a wavelength of 560 nm. The observed values are reported as arbitrary absorbance units (U.A).

The base oxidation level of Nitro Blue Tetrazolium has been normalized to 100 U.A. After the reaction between xanthine and hypoxanthine, which generates the superoxide anion, the oxidation value of Nitro Blue Tetrazolium is significantly increased to 4000 U.A. ca. The presence of ADM-12 in the reaction mixture induces a significant decrease of the oxidation level from a 3 μM concentration, with an efficacy increasing proportionally to the concentration, and inhibiting completely the oxidation (109±37) when evaluated at the concentration of 1 mM.

TABLE 1

| | | ADM-12 | | | | | |
|---|---|---|---|---|---|---|---|
| | | concentration (μM) | | | | | |
| Control | Oxidation | 0.1 | 1 | 3 | 30 | 100 | 1000 |
| 100 ± 16 | 3996 ± 81 | 3753 ± 116 | 3790 ± 70 | 3629 ± 56* | 3611 ± 112* | 2844 ± 42** | 109 ± 37** |

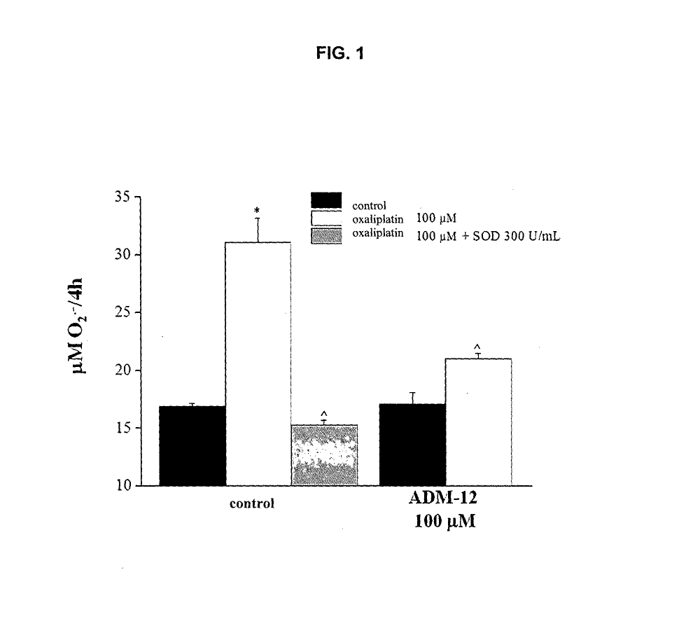

The antioxidant profile of ADM-12 also emerges in a cell culture of primary rat astrocytes (FIG. 1). Incubation of cells with the chemotherapic agent oxaliplatin 100 μM induces a significant production of the superoxide anion, which after 4 hours increases from 16.9±0.3 μM of the control to 31.1±2.1 μM; co-treatment with ADM-12 100 μM reduces the production of oxygen free radicals to 21.0±0.4 μM.

When administered in vivo in rats, ADM-12 is able, after a single administration at the dose of 30 mgkg⁻¹, of reverting the hyperalgesia induced by the neurotoxic compound oxaliplatin (2.4 mg kg⁻¹ i.p. for 21 days). ADM-12 is effective after 15 min from the administration per os, reverting the algic values to the control values (Tabella 2).

TABLE 2
EVALUATION OF THE EFFECTS OF ADM-12
ON THE HYPERALGESIA INDUCED BY OXALIPLATIN
IN THE PAW PRESSURE TEST

| | algic treshold to a mechanical stimulus (g) | | | | | |
|---|---|---|---|---|---|---|
| | Pre test | Pre test | after treatment (min) | | | |
| TREATMENT | day 0 | day 21 | 15′ | 30′ | 45′ | 60′ |
| ADM-12 | 73.3 ± 1.4 | 51.2 ± 0.2* | 70.6 ± 4.8\hat{ } | 63.1 ± 1.2\hat{ } | 51.1 ± 2.4 | 51.9 ± 3.3 |
| 30 mg kg⁻¹ | | | | | | |

*P < 0.01 with respect to the pretest carried out before the treatment with oxaliplatin.
\hat{ }P < 0.01 with respect to the value obtained the 21th day of treatment with oxaliplatin.

ADM-12 presents an excellent security profile after a treatment at the dose of 30 mg kg⁻¹ p.o. repeated daily for 21 days. The hystological test of the kidney (FIG. 2) has shown that the renal corpuscle appears constituted by a normal glomerulus of spherical shape surrounded by a narrow space, the Bowmann space. The interstice formed by normal sections of distal convoluted tubules does not present evidence of inflammation and/or edema.

The analysis of liver reveals that the repeated treatment with ADM-12 does not compromise the hepatic cytoarchitecture, allowing to reveal a compact structure constituted by hepatocytes, regularly disposed in subtle laminas densely packed and well colored by eosin. In FIG. 3, the sinusoids may weakly be observed as subtle spaces located between the laminas of hepatic cells.

1. A compound of formula (I) 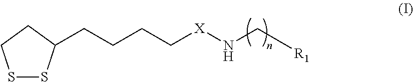
wherein
R1=—SO₃H, —PO₃H, —PO₂(OH)₂, —OPO₂H₂, —NHSO₃H, —S(N═H)Me, —COOH, —SH, —SR or guanidyl;
R═C₁-₄ alkyl, phenyl or 5 or 6 membered aromatic nitrogen heterocycles;
n=1, 2, 3, 4 or 5;
X═C═O, C(OH)H, C(OAlk)H, C═S, CH₂
Alk=C₁₋₆ alkyl linear, branched or cyclic, optionally hydroxylated or polyhydroxylated,
including all possible optical isomers such as enantiomers and/or diastereoisomers, mixtures thereof, either as racemes or in various ratios, and inorganic or organic salts (pharmaceutically acceptable);
excluding compounds N—(R)-lipoyl-β-alanine, N—(R)-lipoyl-β-taurina, N—(R)-lipoyl-aminoethylphosphonic acid, N—(R)-lipoyl-aminoethyldihydrogenphosphate, N-(α)-lipoyl-aminoethanesulfonic acid sodium, potassium, calcium and magnesium salts, N-(α)-lipoyl-6-aminohexanoic acid sodium salt, the sodium salt of N-(α)-lipoyl-␣-amino-n-butanoic acid and the sodium salt of N-(α)-lipoylglycine.

2. The compound according to claim 1 in which R1=—SO₃H.

3. The compound according to claim 1 in which n=2, 3 or 4.

4. The compound according to claim 1 in which X═C═O.

5. A method of medically treating a patient in need thereof, said method comprising: administering a therapeutically effective amount of a compound having formula (I) 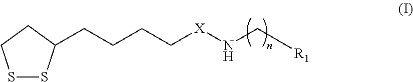
where
R1=—SO₃H, —PO₃H, —PO₂(OH)₂, —OPO₂H₂, —NHSO₃H, —S(N═H)Me, —COOH, —SH, —SR or guanidyl;
R═C₁₋₄ alkyl, phenyl or 5 or 6 membered aromatic nitrogen heterocycles;
n=1, 2, 3, 4 or 5;
X═C═O, C(OH)H, C(OAlk)H, C═S, CH₂
Alk=C₁₋₆ alkyl linear, branched or cyclic, optionally hydroxylated or polyhydroxylated,
including all possible optical isomers such as enantiomers and/or diastereoisomers, mixtures thereof, either as racemes or in various ratios, and inorganic or organic salts; excluding of compounds N—(R)-lipoyl-β-alanine, N—(R)-lipoyl-β-taurina, N—(R)-lipoyl-aminoethylphosphonic acid, N—(R)-lipoyl-aminoethyldihydrogenphosphate.

6. A method of medically treating a patient in need of analgesic, anti-hyperalgesic or anti-allodynic treatment, said method comprising: administering a therapeutically effective amount of a compound having formula (I) 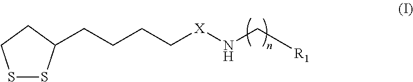
where
R1=—SO₃H, —PO₃H, —PO₂(OH)₂, —OPO₂H₂, —NHSO₃H, —S(N═H)Me, —COOH, —SH, —SR or guanidyl;
R═C₁₋₄ alkyl, phenyl or 5 or 6 membered aromatic nitrogen heterocycles;
n=1, 2, 3, 4 or 5;
X═C═O, C(OH)H, C(OAlk)H, C═S, CH₂
Alk=C₁₋₆ alkyl linear, branched or cyclic, optionally hydroxylated or polyhydroxylated,
including all possible optical isomers such as enantiomers and/or diastereoisomers, mixtures thereof, either as racemes or in various ratios, and inorganic or organic salts.

7. The method according to claim 6 in which R1=—SO₃H.

8. The method according to claim 6 in which n=2, 3 or 4.

9. The method according to claim 6 in which X═C═O.

10. The method according to claim 6, wherein the patient is in need of treatment of neuropathic pain, of pain induced by chemotherapies, pain induced by inflammation of trigeminal nerve, including headache, “restless legs” syndrome, itch or rhinitis.

11. A pharmaceutical composition comprising a compound of formula (I) according to claim 1 and at least another pharmaceutically acceptable ingredient.

12. A pharmaceutical composition according to claim 11 in which a chemotherapic agent is also present.

13. The composition according to claim 12 in which said chemotherapic is neurotoxic, preferably selected from the group consisting of oxalilplatin, cisplatin, paclitaxel, vincristina and vinblastina.

14. A process for the preparation of compounds of formula (I) according to claim 1, the aforesaid process comprising:
contacting lipoic acid or a derivative thereof with a compound of formula (II):
R₁—(CH₂)n-NH₂ (II)
in which
R1=—SO₃H, —PO₃H, —PO₂(OH)₂, —OPO₂H₂, —NHSO₃H, —S(N═H)Me, —COOH, —SH, —SR or guanidyl;
R═C₁₋₄ alkyl, phenyl or 5 or 6 membered aromatic nitrogen heterocycles;
n=1, 2, 3, 4 or 5.
and in which R1, for synthetic convenience, can be appropriately masked or protected.

15. The process according to claim 14 wherein an intermediate of formula (III) is used 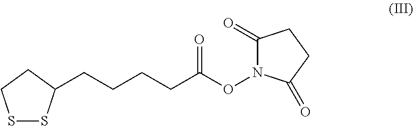 [SMILES: O=C(CCCCC1CCSS1)ON1C(=O)CCC1=O]
including the two possible enantiomers and mixtures thereof.

16. A method of preparing a compound of formula (I) according to claim 1, wherein Use of a compound of formula (III) 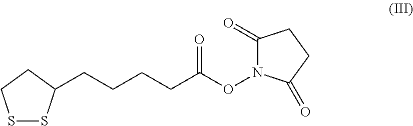 [SMILES: O=C(CCCCC1CCSS1)ON1C(=O)CCC1=O]
is used as a synthetic intermediate.

17. A method of preparing a compound of formula (I) according to claim 1 in which X═C(OH)H, C═S, CH₂, wherein a compound of formula (I) in which X═C═O is used as a synthetic intermediate.
